(19) United States
(12) Patent Application Publication
(10) Pub. No.: US 2011/0130311 A1
(43) Pub. Date: Jun. 2, 2011

(54) METHOD AND SYSTEM FOR DIAGNOSING VIRUS

(75) Inventors: Tae Sung Jung, Gyeongsangnam-do (KR); Seong Won Nho, Gyeongsangnam-do (KR); Ho Bin Jang, Gyeongsangnam-do (KR); In Seok Cha, Gyeongsangnam-do (KR); Sung Bin Park, Gyeongsangnam-do (KR)

(73) Assignee: Industry-Academic Cooperation Foundation Gyeong National University, Jinju-si, Gyeongsangnam-do (KR)

(21) Appl. No.: 12/993,005

(22) PCT Filed: Dec. 22, 2008
(86) PCT No.: PCT/KR08/07568

§ 371 Date: Nov. 16, 2010

(30) Foreign Application Priority Data
Jul. 23, 2008 (KR) 10-2008-0071404

(51) Int. Cl. C40B 60/12; C12Q 1/70

(52) U.S. Cl. 506/39; 435/5

(57) ABSTRACT

Disclose herein is a method for diagnosing virus, comprising the steps of: (a) collecting a sample and lysing virus present in the sample; (b) treating the lysed sample with a specific protease to digest a protein in the sample into peptides; (c) measuring the masses of the peptides in the sample with a mass measurement device; and (d) comparing the masses of the peptides in the sample to the masses of peptides derived from known viral proteins digested with the same protease as used in step (b), thus identifying the protein from which the peptides of the sample were derived. Also disclosed is a system for diagnosing virus which can be used to carry out the above diagnostic method.

### TECHNICAL FIELD

The present invention relates to a method for diagnosing virus, comprising the steps of: (a) collecting a sample and lysing virus present in the sample; (b) treating the lysed sample with a specific protease to digest a protein in the sample into peptides; (c) measuring the masses of the peptides in the sample with a mass measurement device; and (d) comparing the masses of the peptides in the sample to the masses of peptides derived from known viral proteins digested with the same protease as used in step (b), this identifying the protein from which the peptides of the sample were derived. Also, the present invention relates to a system for diagnosing virus which can be used to can out the above diagnostic method.

### BACKGROUND ART

The term “virus” refers to an infectious agent which is made up of a genetic material, such as DNA or RNA, and protein. It generally has a size between 10 nm and 1000 nm depending on the kind thereof. Because it cannot be reproduced by itself, it injects its DNA or RNA into a. host cell, such as a bacterial, plant or animal cell, and then uses the organelle of the host cell to reproduce new viruses. For this reason, the host cell is destroyed.

Some viruses when infected a. host do not cause disease according to the kind of virus and the immune state of the host, whereas some viruses when infected a host are rapidly replicated and distributed, leading to the death of the host or causing vast economic damage. Recently, the highly pathogenic avian influenza (Al) virus N5N1 which is a significant risk factor in terms of zoonosis in Korea and foreign countries showed very high mortality. Specifically, since 2003, the virus H5N1 infected 330 persons in 14 countries, including China, Thailand, Vietnam and Indonesia, leading to 240 deaths. In order to establish immediate therapeutic and preventive countermeasures against diseases caused by these viruses, a very rapid and accurate diagnosis of the diseases is required. However, diagnostic methods capable of accurately differentially diagnosing these diseases are insufficient, and thus these diseases are causing vast economic damage.

Viral diagnostic methods which are currently used to detect viruses and viral proteins and nucleic acids include in-ovo, injection and immunochromatography. Specifically, the in-ovo injection method has high detection sensitivity, but requires an experimental period of 2-5 days in order to detect viruses. Meanwhile, the diagnostic method employing immunochromatography enables viral proteins to be detected in a short time, but has a disadvantage of low detection sensitivity. Due to this advantage, it is not suitable for precise diagnosis, even though it can be used for simple diagnosis. In addition, serological test methods which are used to detect viruses include haemagglutination inhibition (HI) tests and ELISA reactions, but these diagnostic methods require additional experiments in order to examine pathogenicity.

Furthermore, in the case of RT-PCR methods which are widely used to diagnose viruses and which use a primer binding specifically to each viral gene to determine the presence or absence of the viral gene depending on whether a polymerization reaction occurs, false negative results can occur when contaminants, such as polysaccharides and salts, and low/high-pathogenic viruses coexist in a sample extracted from the feces or organs of a subject to be tested. In addition, it is difficult to distinguish pathogenic strains from non-pathogenic strains according to primers which are used in RT-PCR.

### DISCLOSURE

### Technical Problem

The present inventor has found that virus in a virus- containing sample can be rapidly and accurately identified by lysing the sample, measuring the masses of peptides in the lysed sample with a mass spectrometer and comparing the measured peptide masses with the masses of peptides derived from known viral proteins, thereby completing the present invention.

Accordingly, one aspect of the present invention is to provide a method for diagnosing virus. comprising the steps of; (a) collecting a sample and lysing virus present in the sample; (b) treating the lysed sample with a specific protease to digest a protein in the sample into peptides; (c) measuring the masses of the peptides in the sample with a mass measurement device; and (d) comparing the masses of the peptides in the sample to the masses of peptides derived from known viral proteins digested with the same protease as used in step (b), thus identifying the protein from which the peptides of the sample were derived.

Another aspect of the present invention is to provide a system for diagnosing virus, comprising: a database in which information on the masses of peptides derived from viral proteins is stored; a mass measurement device for measuring the masses of peptides in a sample; and a viral protein matching/filtering unit which performs filtering of information in the database, which matches information on the viral protein of the sample, by comparing information on the measured peptide masses of the sample to information on the peptide masses of the database, thus determining information derived from the measured peptide masses of the sample.

### Technical Solution

In one aspect, the present invention relates to a method for diagnosing virus, comprising:
(a) collecting a sample and lysing virus present in the sample;
(b) treating the lysed sample with a specific protease to digest a protein in the sample into peptides;
(c) measuring the masses of the peptides in the sample with a mass measurement device; and
(d) comparing the masses of the peptides in the sample to the masses of peptides derived from known viral proteins digested with the same protease as used in step (b), thus identifying the protein from which the peptides of the sample were derived. Hereinafter, each step of the method for diagnosing virus will be described in further detail.

Step (a): Collection and Lysis of Sample

As used herein, the term “sample” is meant to include samples collected from the sites of frequent occurrence of viral infection (e.g., blood, tissue, phlegm, urine, feces, etc.), cell-cultured samples, and samples obtained in nature. The sample can be collected using any method known in the art. The collected sample is preferably subjected to homogenization and serial dilution. If the sample thus collected contains many impurities in addition to virus to be diagnosed, a step of separating virus from the sample may additionally be carried out to remove these impurities. To carry out the step of separating virus from the sample, any method known in the art may be used as long as it can separate virus from the sample. In an embodiment of the present invention, a method utilizing the antibodies, resins or beads capable of adsorbing various small peptides may be used to separate virus from the sample. An example of the virus separation method is immuno-magnetic separation (IMS).

The sample may be lysed with a lysis buffer without any further treatment. Alternatively, virus separated from the sample may be treated with a lysis buffer or lysed by sonication or heat treatment. In an embodiment of the present invention, virus separated from the sample is treated with a lysis buffer. For this purpose, the lysis buffer preferably contains Triton X-100 and DL-dithiothreitol (DTT), Triton-100 that is a nonionic surfactant serves to increase the permeability of the cell membrane, and thus is useful for lysing virus, and DTT serves to digest disulfide bonds in a three-dimensional protein structure, such that the difficulty in access of enzyme caused by the three-dimensional structure can be alleviated, The lysis buffer which is used in the present invention may additionally contain NaCl and Tris-HCl. Through this step, virus present in the sample will be lysed.

In the present invention, although step (b) may be carried out immediately after step (a), a step of filtering the lysed sample may also be added between steps (a) and (b). By this filtering step, the constituent components of the buffer used in the lysis step can be removed from the lysate, and the analysis of the lysate by a mass measurement device can be facilitated, while only pure disrupted virions can be obtained.

Step (b): Treatment with Protease

As is well known in the art, protease degrades proteins into peptides. This step is preferably carried out under microwave irradiation. The wavelength and high temperature generated by microwave irradiation induce protein-protein collision and molecule-molecule collision to facilitate the digestion of viral proteins by protease, and can shorten the time required for protein digestion by inhibiting protein aggregation, that is, protein recombination, which can occur during protein digestion. Also, this microwave irradiation can shorten the reaction time by eliminating the need for an alkylation process which must have been carried out to prevent recombination of disulfide bonds digested by a compound (e.g., DTT) which is used to disrupt the three-dimensional structure of a protein so as to facilitate access of protease to the active site of the protein.

Step (c): Measurement of Masses of Peptides

The present invention is characterized in that the masses of peptides obtained by degrading the virus (that is, the total protein of the virus) by protease are measured with a mass measurement device. As the mass measurement device, a mass spectrometer is preferably used. In the present invention, MALDI-TOF MS (matrix assisted laser desorption/ionization—time of flight mass spectrometry can be used for mass analysis, For example, a Voyager De STR MALDI-TOF MS instrument can be used. However, various types of mass spectrometry (MS) and MS/MS, which allows for the measurement of proteins, may be used in the present invention, Herein, as the matrix, sinapic acid may, for example, be used, Factors influencing detection efficiency include: (1) delay time (DE)—the time interval nanoseconds) between one laser irradiation and the following one; (2) grid voltage (%)—the magnitude of energy required for peptide ions to fly in a MALDI-TOF MS tube until they are detected in a detector; (3) mass range—the mass range of peptides to be detected; and (4) laser intensity—laser irradiation dose. Such factors can be suitably determined by a person skilled in the art.

Step (d) Comparison of Measured Peptide Masses With Known Peptide Masses

The masses of peptides derived from known viral proteins can be obtained, for example, as follows. Protease is an enzyme that recognizes an amino acid sequence and cuts the recognition site. Thus, it is possible to infer the sequences of peptides which can be obtained when a viral protein consisting of a specific amino acid sequence is treated with a specified protease, and the masses of the peptides can be calculated with reference to the known mass of the amino acid sequence. Based on the calculated masses of the peptides derived from the known viral proteins, the virus in the sample can be identified by screening peptides having masses which are identical or most similar to the measured peptide masses and identifying the viral protein from which the peptides having the masses were derived.

In addition, step (d) may be carried out by determining whether there exists a set of masses of peptides in the sample, which matches a set of masses of peptides present specifically in the known viruses digested with the protease.

The viral diagnostic method according to the present invention can be carried out using a system having the following construction.

In another aspect, the present invention relates to a system for diagnosing virus, comprising; a database in which information on the masses of peptides derived from viral proteins is stored; a mass measurement device for measuring the masses of peptides present in a sample; and a viral protein matching/filtering unit which performs filtering of information in the database, which matches information on the viral protein of the sample, by comparing information on the measured peptide masses of the sample to information on the peptide masses of the database, thus determining information derived from the measured peptide masses of the sample.

In the present invention, the mass measurement device is preferably, but not limited to a mass spectrometer.

The database which is used in the present invention can be structured such that it stores theoretical masses obtained by calculating the masses of peptides which can be obtained when a specific viral protein is degraded by a specified protease on the basis of genetic information (an amino sequence or a base sequence). Herein, the database is structured such that the masses of the peptides are connected with information on the name of the viral protein from which the peptides were derived, and the name of protease used. The kinds of viral proteins and proteases to be included in the database of the present invention are not limited. The database of the present invention can also be expanded by including information on unknown viruses, proteins and proteases in future.

The viral diagnostic system of the present invention may additionally comprise a sample receiving unit and a protease storing unit for introducing protease into the sample receiving unit. The sample receiving unit may additionally comprise a microwave source for irradiating the sample receiving unit with microwaves.

Also, the viral diagnostic system of the present invention may additionally comprise a viral information output unit which outputs information on virus contained in a sample on the basis of information on an extracted viral protein. If the number of information on virus contained in the sample, the viral information output unit can output each information separately. In addition, in the viral diagnostic system of the present invention, the database, the mass measurement device, the viral protein extraction unit and the viral information output unit are preferably connected with each other through a network.

### Advantageous Effects

According to the present invention, virus can be rapidly and accurately diagnosed. Particularly, in the present invention, a sample is irradiated with microwaves during a process of degrading the total protein of the virus, to be diagnosed, by protease, such that the time required for protein degradation into peptides can be significantly shortened, and thus the time required for diagnosing the virus can also be shortened. Because virus is diagnosed using the total protein of the virus rather than using only some proteins remaining after liberating non-structural proteins from the virus after lysis of the virus, the present invention can be used for various analytical applications, including the serotype analysis and pathogenic analysis of virus and the analysis of vaccine virus, In addition, it is possible to diagnose human, animal and plant viruses in the same or similar manner.

### DESCRIPTION OF DRAWINGS

FIG. 1 shows the mass spectra of peptides measured with a mass spectrometer according to the present invention.

FIG. 2 shows the mass values of peptides measured with a mass spectrometer according to the present invention, the mass values being presented in the form of an excel file.

FIG. 3 shows a process of substituting the mass values of peptides, measured with a mass spectrometer according to the present invention, into a diagnostic system of the present invention.

FIG. 4 shows the results diagnosed using a diagnostic system of the present invention, the results indicating that Newcastle disease virus was diagnosed with the highest concordance.

FIG. 5 shows the results diagnosed using a diagnostic system of the present invention, the results indicating that Newcastle disease virus was diagnosed with the highest concordance.

FIG. 6 shows the results diagnosed using a diagnostic system of the present invention, the results indicating that Newcastle disease virus was diagnosed with the highest concordance.

### BEST MODE

### Example 1

### Collection of Sample

Samples were collected from the tissue, blood, phlegm, urine and feces of a chicken suspected of being infected with Newcastle virus. The collected samples were homogenized, diluted serially and used in the following Example 2.

### Example 2

### Lysis

Virus was separated from the samples, and 10 μl (containing 4.811 μg virus/μl) of an aliquot of the virus was transferred to an e-tube. 20 μl of a viral lysis buffer [containing 1% Triton X-100 (Amresco), 2 mM DTT (DL-dithiothreitol, Promega) 150 mM NaCl (Amresco) and 10 mM Tris-HCl (Amresco), pH 7.4] was added thereto, and the mixture was incubated at room temperature for 15 minutes.

Before the use of Microcon as described below, 470 μl of resin water was added in order to determine the maximum concentration.

### Example 3

### Filtration of Lysate

A micron centrifugal filter device (YM-3; nominal molecular weight limit: 3,000 Daltons; Amicon) was used to separate only pure disrupted virions from the reaction solution consisting of the mixture of disrupted virions and virus-disrupting buffer, obtained in Example 2.

### Example 4

### Degradation of Protein by Microwave Irradiation

25 μl of 10 mM DTT was added to 10 μl of the virion solution obtained in Example 3 and was slightly mixed for 20 minutes. 20 μl of trypsin solution (20 μg/ml trypsin in 50 mM NH₄HCO₃) was added thereto, and then the cap of the e-tube was perforated with a syringe needle to form 3-4 holes. Then, the mixed solution was irradiated with microwaves in a domestic microwave oven (SAMSUNG RE-442B) for 10 minutes. After irradiation, the solution was cooled at 4° C. for 5 minutes, and then 20 μl of a calibrant (insulin from bovine pancreas; FW=5733; SIGMA) was added.

### Example 5

### Diagnosis of Virus by Inventive Method

A. MALDI-TOF ionization plate (ID: 100)

As a matrix, sinapic acid (Fluka) was used, and a sandwich method was used to target the viral protein on a plate.

Specifically, 40 μl of 25% trifluoroacetic acid (TFA; MERCK) was mixed with 960 μl of resin water to prepare 1% TFA solution. 500 μl of the 1% TFA solution was mixed with 500 μl of acetonitrile (CAN; 99.93+% HPLC grade; Sigma Aldrich), and 20 mg of sinapic acid (Fluka) was added thereto. The mixed solution was vortexed for 30 minutes, and 1 μl of the vortexed solution was dropped onto an ionization plate, and then dried in air. Then, 1 μl of the sample solution obtained in Example 4 was dropped onto the ionization plate. Also, 1 μl of a solution obtained by mixing 990 μl of acetone (99+% HPLC grade, Sigma Aldrich) with 10 μl of resin water, adding 200 ml of SA and vortexing the mixed solution was dropped onto the ionization plate, and then dried in air.

B. Carrying out of MAILDI-TOP MS

The mass spectra of the peptides present in the sample solution were obtained by carrying out MALDI-TOF MS using a Voyager DE STR MALDI-TOF MS reflector mode under the following conditions: delay time (DE): 100 ns; grid voltage (%): 68%; mass range: 800-10000; and laser intensity: 2205.

### Mode for Invention

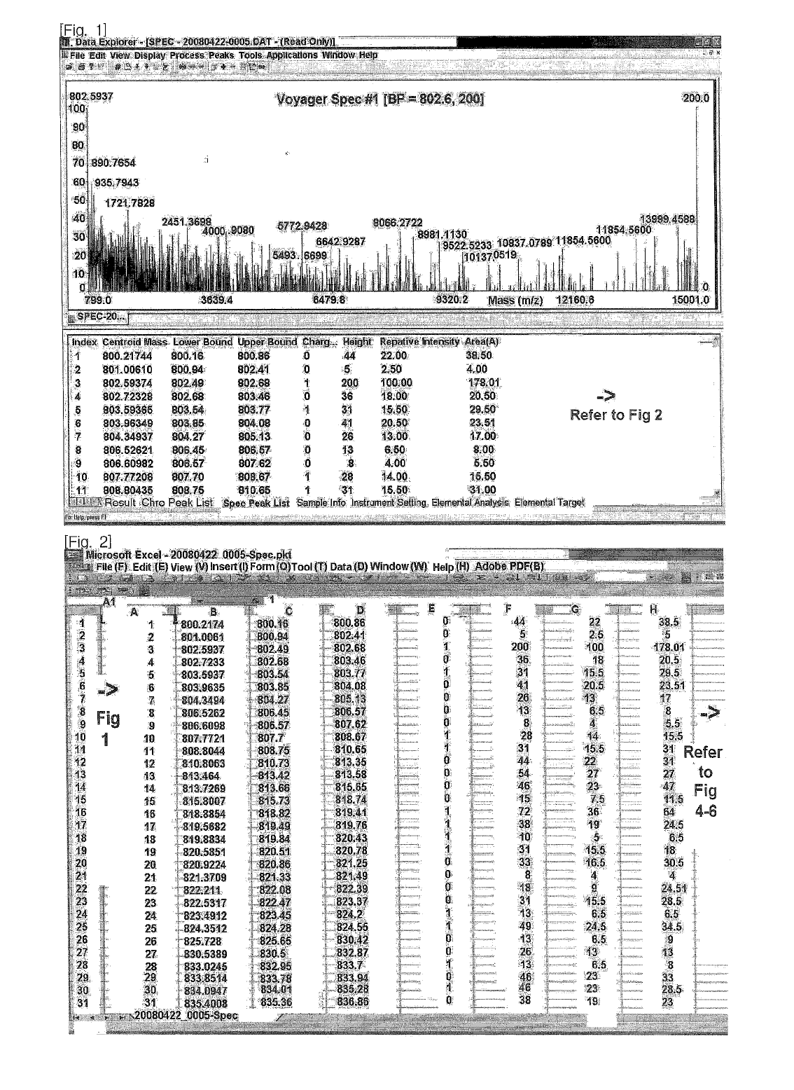

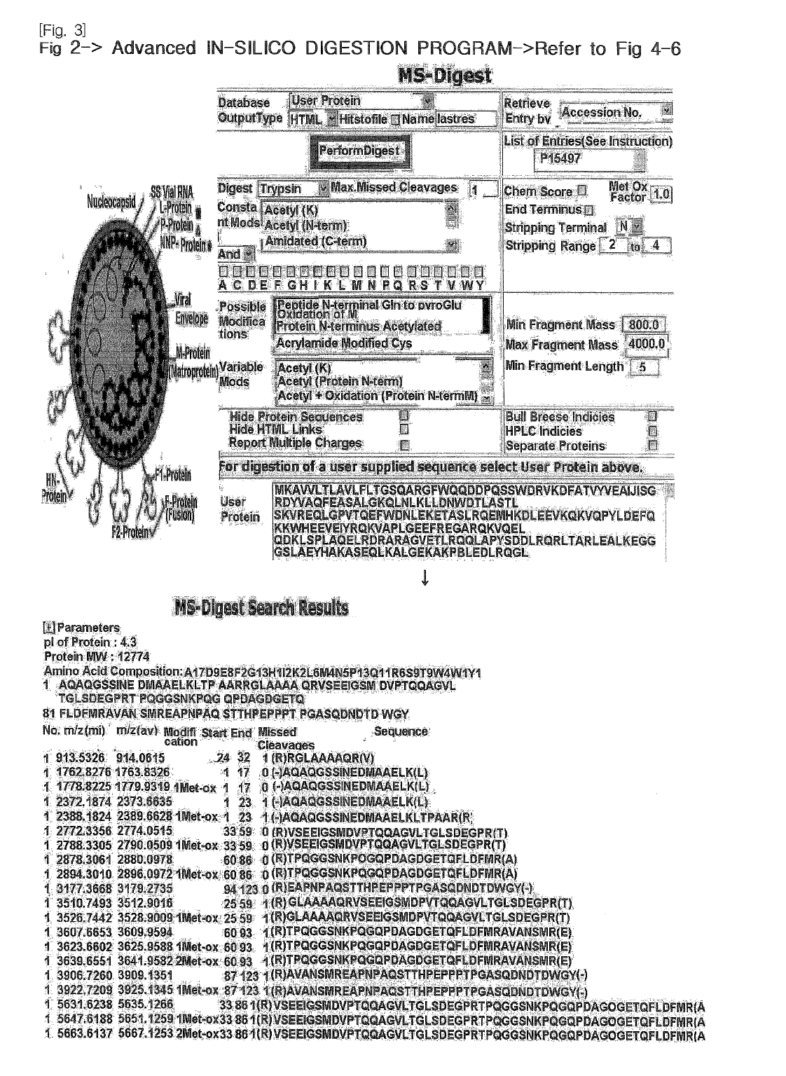

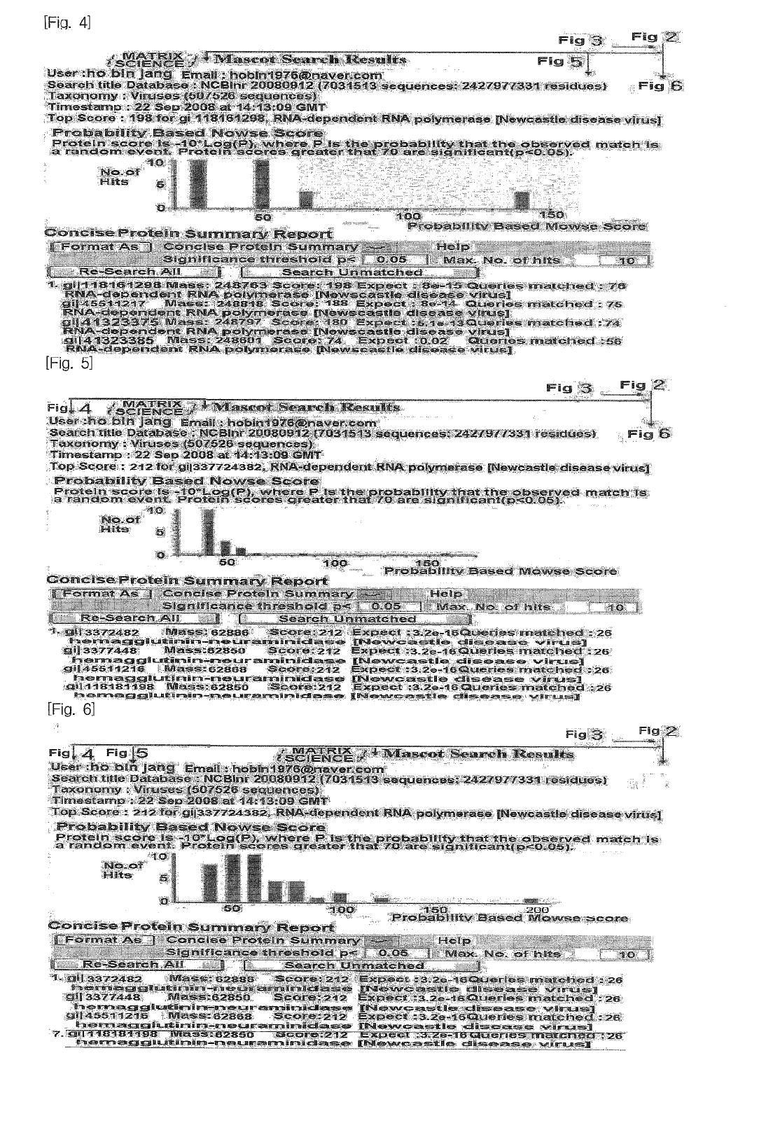

The mass values of peptides measured in Example 5 were obtained in the form of mass spectra (FIG. 1)) and an excel file (FIG. 2), and then the measured mass values were substituted into the diagnostic system of the present invention to carry out the diagnosis of virus present in the sample (FIG. 3). hr the diagnostic results, the viral proteins in the sample were diagnosed as the fusion protein of Newcastle disease virus (FIG. 4), the matrix protein of Newcastle disease virus (FIG. 5) and the RNA-dependent RNA polymerase of Newcastle disease virus (FIG. 6), suggesting that the virus in the sample could be accurately diagnosed as Newcastle disease virus.

### INDUSTRIAL APPLICABILITY

According to the present invention, virus can be rapidly and accurately diagnosed. Particularly, in the present invention, a sample is irradiated with microwaves during a process of degrading the total protein of the virus, to be diagnosed, by protease, such that the time required for protein degradation into peptides can be significantly shortened, and thus the time required for diagnosing the virus can also be shortened. Because virus is diagnosed using the total protein of the virus rather than using only some proteins remaining after liberating non-structural proteins from the virus after lysis of the virus, the present invention can be used for various analytical applications, including the serotype analysis and pathogenic analysis of virus and the analysis of vaccine virus. In addition, it is possible to diagnose human, animal and plant viruses in the same or similar manner.

1. A method for diagnosing virus. comprising the steps of;
(a) collecting a sample and lysing virus present in the sample;
(b) treating the lysed sample with a specific protease to digest a protein in the sample into peptides;
(c) measuring the masses of the peptides in the sample with a mass measurement device; and
(d) comparing the masses of the peptides in the sample to the masses of peptides derived from known viral proteins digested with the same protease as used in step (b), thus identifying the protein from which the peptides of the sample were derived.

2. The method of claim 1, wherein the lysis of the virus in step (a) is carried out after separating the virus from the sample.

3. The method of Claim I, wherein the lysis of the virus in step (a) is carried out by treating the virus with a lysis buffer containing DTT and Triton X-100 as essential components.

4. method of claim 3, additionally comprising, after step (a), a step of filtering the lysed sample to remove the components of the lysis buffer.

5. The method of claim 1, wherein step (b) is carried out while irradiating the sample with microwaves.

6. The method of claim 1, wherein the mass measurement device in step (c) is a mass spectrometer.

7. method of claim 1, wherein step (d) is carried out by determining whether there exists a set of masses of peptides in the sample, which matches a set of masses of peptides present specifically in the known viruses digested with the protease.

8. A system for diagnosing virus, comprising:
a database in which information on the masses of peptides derived from viral proteins is stored;
a mass measurement device for measuring the masses of peptides in a sample; and
a viral protein matching/filtering unit which performs filtering of information in the database, which matches information on the viral protein of the sample, by comparing information on the measured peptide masses of the sample to information on the peptide masses of the database, thus determining information derived from the measured peptide masses of the sample.

9. The system of claim 8, wherein the mass measurement device is a mass spectrometer.

10. The system of claim 8, additionally comprising:
a sample receiving unit; and
a protease storing unit for introducing protease into the sample receiving unit.

11. The system of claim 10. wherein the sample receiving unit additionally comprises a microwave source for irradiating the sample receiving unit with microwaves.

12. The system of claim 8, additionally comprising a viral information output unit which outputs information on virus contained in the sample on the basis of information on an extracted viral protein.

13. The system of claim 12, wherein, if the number of information on the virus contained in the sample is two or more, the viral information output unit outputs each information separately.

14. The system of claim 8, wherein the database, the mass measurement device, the viral protein extraction unit and the viral information output unit are connected with each other through a network.
